(19) United States
(12) Patent Application Publication
(10) Pub. No.: US 2023/0098195 A1
(43) Pub. Date: Mar. 30, 2023

(54) METHOD OF DETERMINING PERCENTAGE OF IMMUNE CELL TYPES IN A SALIVA SPECIMEN

(71) Applicant: Tru Diagnostics, Inc., Lexington, KY (US)

(72) Inventors: Andrew E. TESCHENDORFF, Shanghai (CN); Ryan M. SMITH, Lexington, KY (US)

(21) Appl. No.: 17/490,160

(22) Filed: Sep. 30, 2021

(51) Int. Cl. G01N 33/50; C12Q 1/6881

(52) U.S. Cl. CPC G01N 33/5047; C12Q 1/6881; C12Q 2500/00

(57) ABSTRACT

A method of determining a percentage of cell-types in a saliva specimen includes the steps of obtaining genomic DNA from the saliva specimen, observing cytosine methylation at specific CG loci in the genomic DNA of the saliva specimen, comparing the observed methylation with the methylation observed in genomic DNA collected from a reference group of saliva specimens and correlating the CG loci methylation observed in the genomic DNA of the saliva specimen with the methylation observed in the genomic DNA of the reference group of saliva specimens to determine the percentage of cell-types in the saliva specimen.

### TECHNICAL FIELD

This document relates generally to the construction and validation of a DNA methylation reference for saliva tissue that is useful for estimating cell-type fractions in a saliva specimen for which a DNA methylation profile has been generated.

### BACKGROUND

This document relates to a method of determining a percentage of cell-types in a saliva specimen and, more particularly, to determining the percentage of eight different cell types in a human saliva specimen. Those eight cell types include: epithelial cells, and the immune cell types: neutrophils, eosinophils, monocytes, CD4+ and CD8+ T-cells, natural (NK) killer cells and B-cells. This method is an important preliminary step in the construction of a method of biological age prediction.

Aging may be defined as the progressive loss of cell function. Loss of cell function is evidenced in many ways including, for example, gaining wrinkles (skin losing elasticity), worsening eyesight and loss of range of motion of the extremities.

Many aspects of cell function are controlled by gene expression. DNA methylation (DNAm)—the epigenetic marker that is measured and observed in the current method—is a chemical modification of the genome that is able to control gene expression. That modification is usually found as part of the dinucleotide CpG. The current method utilizes a DNA methylation reference matrix for human saliva that identifies which CpG sites in the human genome are connected to signs of aging and disease development.

By analyzing the pattern of DNA methylation in the genome of a human subject with our state-of-the-art algorithm, we can check if the human subject's biological systems are aging faster or slower than the average person of the same chronological age.

It is known that DNA methylation changes in response to a wide range of environmental factors including, but not necessarily limited to, nutrition, exercise habits, medications, injuries, environmental pollutions, amount of sleep enjoyed and amount of stress endured. With the biological age information provided by the current method, it is possible for a human subject to make lifestyle changes to slow biological aging and monitor those results. This allows the human subject to receive positive feedback from good lifestyle changes and enjoy a longer and healthier life.

### SUMMARY

In accordance with the purposes and benefits set forth herein, a new and improved method is provided for determining a percentage of cell-types in a saliva specimen. That method comprises the steps of: (a) obtaining genomic DNA from the saliva specimen; (b) observing cytosine methylation at a plurality of CG loci in the genomic DNA of the saliva specimen, wherein said observing includes performing a bisulfate conversion process on the genomic DNA of the saliva specimen so that cytosine residues in the genomic DNA of the saliva specimen are transformed to uracil, while 5-methylcytosine residues in the genomic DNA of the saliva specimen are not transformed to uracil;

(c) comparing the CG loci methylation observed in (b) to the CG loci methylation observed in genomic DNA collected from a reference group of saliva specimens; and (d) correlating the CG loci methylation observed in (b) with CG loci methylation observed in the reference group of saliva specimens to determine the percentage of cell-types in the saliva specimen.

In one possible embodiment of the method, the plurality of CG loci in the DNA of the human subject includes 157 CG loci identified as follows: cg24462702, cg25599673, cg11944101, cg02661764, cg1040649, cg24662823, cg01874152, cg16193207, cg25486399, cg10240150, cg07713946, cg07960083, cg11848483, cg21224730, cg10864951, cg25226014, cg12032198, cg13750061, cg03274669, cg19994968, cg24612198, cg08450017, cg06164961, cg00219921, cg11531557, cg02324835, cg05356800, cg22999502, cg08622923, cg22512531, cg24456340, cg04759756, cg14094409, cg10977115, cg11583544, cg04042333, cg26518932, cg11804414, cg26757673, cg13988440, cg07728874, cg16039157, cg10480329, cg16636767, cg13781869, cg22675702, cg11664417, cg13430807, cg00208012, cg24788483, cg25517015, cg26326621, cg02780988, cg05923857, cg20641531, cg24365417, cg10278149, cg14976569, cg26997966, cg12655112, cg12873119, cg12207930, cg12037947, cg27366072, cg26538782, cg15035590, cg14936008, cg20452738, cg05205074, cg20602300, cg04838847, cg17232476, cg18664915, cg13823257, cg24777505, cg05579731, cg23683962, cg23730277, cg00867406, cg19276014, cg22281206, cg07721872, cg02087075, cg17304531, cg04289069, cg03730703, cg14060402, cg14331899, cg13617280, cg05875239, cg03538296, cg11335172, cg15956469, cg15241779, cg10628126, cg27309871, cg06202778, cg18310515, cg10142452, cg21474838, cg01940139, cg03605454, cg21333217, cg11100450, cg03708221, cg15052335, cg02150910, cg11637084, cg03886681, cg07307830, cg26227465, cg13468144, cg03146219, cg25600606, cg01040749, cg23599224, cg15520845, cg10611016, cg21792134, cg12809098, cg11597277, cg10487428, cg00084577, cg17080697, cg17932662, cg26105956, cg09163720, cg13079571, cg09859659, cg00471371, cg01718139, cg07356342, cg22381196, cg20720686, cg20240243, cg04476877, cg05078091, cg01953317, cg07050712, cg22543648, cg10454864, cg26103369, cg18966140, cg08077807, cg21510284, cg02494549, cg26156120, cg21261709, cg06620016, cg14320120, cg02031326, cg00059879, cg02195201, cg07033790, cg25270424, cg16760382, and cg20263733.

In another possible embodiment of the method, the plurality of CG loci in the DNA of the human subject includes 64 CG loci identified as follows: cg08846870, cg25139229, cg18982286, cg20820767, cg01657186, cg06457736, cg08804626, cg13496041, cg01311222, cg22216196, cg22935422, cg04869379, cg10818657, cg06055229, cg00715197, cg01062942, cg09958560, cg24757533, cg06267617, cg10257110, cg03331514, cg11586930, cg19082559, cg13493526, cg08101036, cg20944964, cg06830450, cg24002183, cg05258935, cg23218363, cg13189207, cg12050271, cg07380416, cg17936488, cg24749672, cg20695297, cg15046675, cg08075204, cg09638208, cg16321975, cg09354037, cg16509569, cg14556909, cg18555277, cg02794695, cg22801799, cg20425130, cg25666403, cg02026204, cg09153080, cg07420137, cg01951274, cg17127769, cg11528914, cg12253437, cg26232412, cg27482619, cg25574765, cg01903374, cg21376733, cg06380123, cg10673833, cg27284288, and cg13165140.

In one or more of the many possible embodiments of the method, the method includes the step of amplifying the genomic DNA of the saliva specimen by a polymerase chain reaction process. In one or more of the many possible embodiments of the method, the step of observing of the cytosine methylation at the plurality of CG loci includes the further steps of hybridizing the genomic DNA to a beadchip to create a genomic DNA hybridized beadchip, staining the genomic DNA hybridized beadchip and then imaging the genomic DNA hybridized beadchip.

In one or more of the many possible embodiments of the method, the method includes the step of using a regression algorithm to correlate the CG loci methylation observed in (b) with the CG loci methylation observed in the reference group of saliva specimens of known cell-type percentages.

In the method, the genomic DNA of the saliva specimen is derived from a human subject. Further, the genomic DNA of the reference group of saliva specimens, that makes up the DNA methylation reference matrix, is derived from a reference group of human individuals.

In the following description, there are shown and described several preferred embodiments of the method of determining the percentage of cell-types in a saliva specimen. As it should be realized, that method is capable of other, different embodiments and its several details are capable of modification in various, obvious aspects all without departing from the method as set forth and described in the following claims. Accordingly, the drawings and descriptions should be regarded as illustrative in nature and not as restrictive.

### BRIEF DESCRIPTION OF THE DRAWING FIGURES

The accompanying drawing figures incorporated herein and forming a part of the specification, illustrate certain aspects of the method and together with the description serve to explain certain principles thereof.

FIG. 1 is a map depicting a 2 cell-type DNA methylation reference matrix for human saliva consisting of 64 CpGs that can easily discriminate squamous epithelial cells (EPI) from immune cells (IC). “0” values indicate low methylation. “1” values indicate full methylation.

FIG. 2 is a scatterplot of the DNA methylation (DNAm) values of the CpGs in each cell-type.

FIG. 3 is a boxplot that displays the DNAm level (Fraction) of one of the CpGs across the buccal swabs (Epi(BUC): high purity>95%, squamous epithelial cells, n=10), epithelial cell lines (Epi(CellL); purity approximately 100% but not squamous-epithelial specific n=11, 42 immune cell samples (IC).

FIG. 4 includes six panels depicting the estimated cell-type fractions (denoted “f” on y-axis) with the cell-type indicated on the x-axis (Epi=epithelial, IC=immune cell), for six corresponding DNA methylation datasets, with the first one (SCM2) containing 4 epithelial cell lines, the next two containing whole blood samples profiled with different Illumina technologies, and the last row displaying the results for the purified neutrophil (NEU), monocytes (MO) and T-cell (Tcell) samples from BluePrint. This figure demonstrates that the DNA methylation reference matrix is accurately predicting the high epithelial or immune cell purity of all these samples.

FIG. 5 includes two panels depicting the estimated epithelial (left panel, x-axis) and immune cell (right panel, x-axis) fractions against the corresponding true fractions (y-axis), as obtained using the DNA reference matrix for saliva on simulated mixtures where random proportions of epithelial cell lines from SCM2 and whole blood samples from the NSHD have been mixed together.

FIG. 6 includes six panels wherein each panel displays the predicted cell-type fraction (y-axis, f) for seven immune cell subtypes (x-axis) in an Illumina 450k set profiling purified samples as indicated in the title of each panel. The first row are for 139 purified neutrophil (Neu) samples, 139 purified monocyte samples (Mono) and 139 purified CD4+ T-cell samples, all derived from BluePrint (BP). The second row are for purified B-cells (n=100 samples), CD4+ T-cells (n-98 samples) and Monocytes (n=104 samples) derived from a case-control study in type-1 diabetes (T1D).

FIG. 7 includes six panels wherein each panel displays the predicted cell-type fraction (y-axis, f) for seven immune cell subtypes (x-axis) in an Illumina EPIC set profiling purified immune-cell samples as indicated in the title of each panel.

FIG. 8 includes seven panels wherein each panel compares the estimated cell-type fraction (x-axis) to the true fraction (y-axis) for each of the seven cell types, as indicated in the title of each panel, as derived from an in-silico mixture experiment where for each cell-type, mixing proportions are derived from the same distribution.

FIG. 9 includes seven panels wherein each panel compares the estimated cell-type fraction (x-axis) to the true cell fraction (y-axis) for each of the seven cell types, as indicated in the title of each panel, and as derived from an in-silico mixture experiment where underlying cell-type fractions are sampled from realistic fractions found in saliva and blood.

Reference will now be made in detail to the present preferred embodiments of the method.

### DETAILED DESCRIPTION

Below is a brief glossary of scientific terms to help understand the ensuing discussion:

### Glossary

DNA methylation (DNAm): a covalent modification of DNA that occurs predominantly at cytosines of CG dinucleotides (“CpGs”), which can regulate the activity of nearby genes and which is cell-type specific.
DNA methylation reference matrix (DMRM): a mathematical matrix object, with rows labeling CpGs and columns labeling cell-types, and with entries representing DNA methylation values, which will differ between cell-types. This latter property allows this reference matrix to be used for cell-type deconvolution.
Cell type deconvolution: a mathematical procedure whereby a genome-wide molecular profile (e.g. a DNA methylation profile) of a bulk tissue sample (e.g. a saliva specimen) is decomposed into components representing the different cell-types in the tissue. This decomposition allows the fractions of these cell-types in the sample to be estimated, and may also allow the identification of DNAm changes that occur only in specific cell-types.

### 1. Construction of a DNA Methylation Reference Matrix for Saliva

Like blood, saliva is a relatively easy tissue to access. DNA from saliva can be readily extracted and used to profile DNA methylation genome-wide using technologies such as Illumina beadarrays. For instance, the EPIC beadarray can measure the methylation status of over 850,000 CpGs in the human genome. What makes saliva potentially more interesting than blood as a source of DNA is the fact that saliva specimens contain epithelial cells in addition to the wide variety of immune-cells that are also found in blood. Saliva may also contain bacterial DNA. Such DNA however would not be measured with technologies such as Illumina beadarrays since the probes of these arrays are designed to hybridize to fragments of human DNA.

Thus, as far as human sources of DNA are concerned, saliva is made up primarily from epithelial cells, and specifically the squamous epithelial cells from the buccal epithelium, as well as the 6-7 main immune cell-types which are also found in blood. These include neutrophils, eosinophils, monocytes, CD4+ and CD8+ T-cells, natural-killer (NK) cells and B-cells. It is worth pointing out at this stage, that depending on the downstream application, one may only be interested in measuring the epithelial fraction in saliva specimens, which means that for all practical purposes, the variety of different immune-cells can be treated as belonging to one broad immune-cell class. While it is plausible that the epithelial cells in saliva may themselves be heterogeneous, depending on which layer from the buccal epithelium they derive from, this heterogeneity has not yet been explored and is likely to be more informative in buccal swabs, which capture a larger proportion of epithelial cells.

Thus, one can in principle build two different DNA methylation reference matrices for saliva:
one defined at the resolution of two main cell-types (epithelial and immune cell), and
one defined at the resolution of 8 cell-types (1 epithelial and 7 immune cell subtypes).

First we will describe the construction of the 2 cell-type DMRM for saliva. To build the DMRM, it is necessary to generate genome-wide DNAm profiles for the underlying cell-types, or to use existing DNAm profiles from the public domain. Here we will use existing DNAm data from the public domain, which we show is perfectly adequate. First, for the immune-cells, the study of Reinius et al (Reinius L E, Acevedo N, Joerink M, Pershagen G, Dahlen S E, Greco D, Soderhall C, Scheynius A, Kere J: Differential DNA methylation in purified human blood cells: implications for cell lineage and studies on disease susceptibility. PLoS One 2012, 7:e41361.) has profiled 6 independent samples for each of the 7 main immune cell subtypes, i.e. a total of 42 samples represented purified cell populations obtained by FACS sorting against well-known immune cell-type markers.

For the epithelial component, we use an iterative strategy. First we use our published DMRM containing DNAm reference profiles for generic epithelial and immune cell types (Zheng S C, Webster A P, Dong D, Feber A, Graham D G, Sullivan R, Jevons S, Lovat L B, Beck S, Widschwendter M, Teschendorff A E: A novel cell-type deconvolution algorithm reveals substantial contamination by immune cells in saliva, buccal and cervix. Epigenomics 2018, 10:925-940.) to estimate epithelial fractions in a large buccal swab study of 790 samples (Teschendorff A E, Yang Z, Wong A, Pipinikas C P, Jiao Y, Jones A, Anjum S, Hardy R, Salvesen H B, Thirlwell C, et al: Correlation of Smoking-Associated DNA Methylation Changes in Buccal Cells With DNA Methylation Changes in Epithelial Cancer. JAMA Oncol 2015, 1:476-485.), aim being to identify buccal swabs of high epithelial purity (epithelial fraction>0.95 i.e. >95%). This strategy is feasible given that the Reinius profiles and buccal-swab study used the same Illumina 450k technology to generate the DNAm profiles. We identified 10 buccal swabs with an epithelial purity higher than 95%, comparable to what could have been achieved via FACS sorting. Then, by comparing the DNAm profiles of the 10 high purity buccal swabs and a set of 11 epithelial cell lines from ENCODE (Gerstein M: Genomics: ENCODE leads the way on big data. Nature 2012, 489:208.), to the 42 immune cell samples from Reinius, we identified those CpGs that best discriminate squamous epithelial from immune-cells, thus allowing us to build a DMRM that is more tailored for saliva, as compared to the one published in Zheng et al (Zheng S C, Webster A P, Dong D, Feber A, Graham D G, Sullivan R, Jevons S, Lovat L B, Beck S, Widschwendter M, Teschendorff A E: A novel cell-type deconvolution algorithm reveals substantial contamination by immune cells in saliva, buccal and cervix. Epigenomics 2018.).

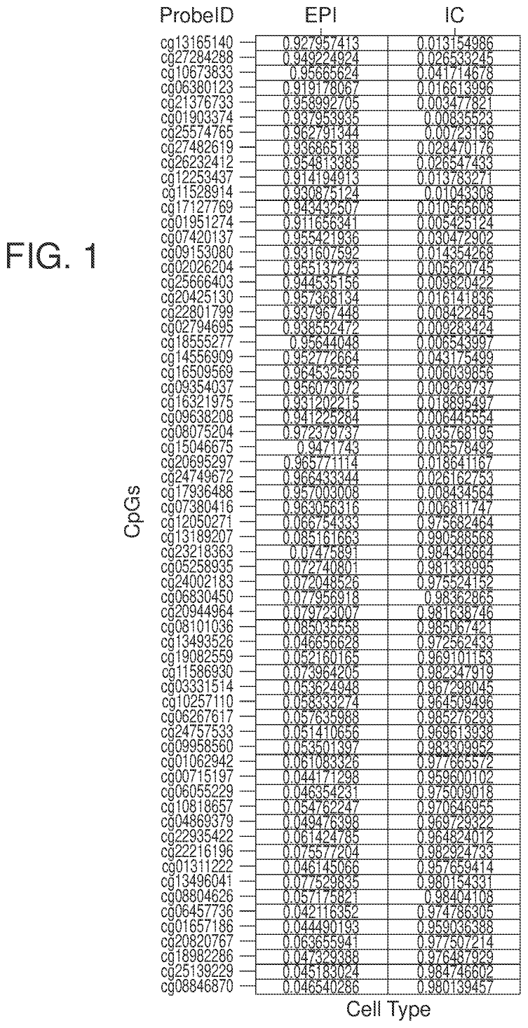

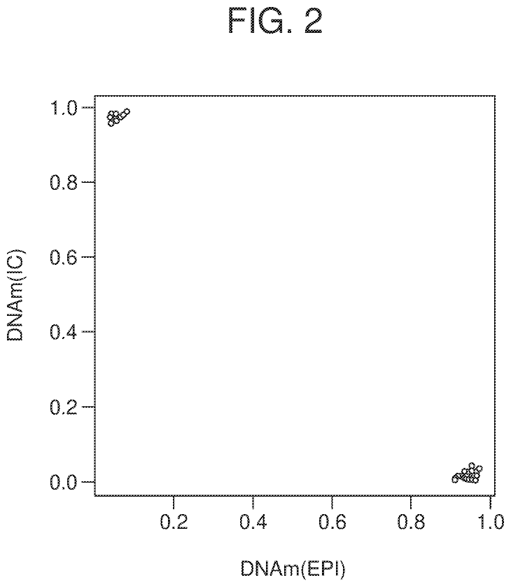

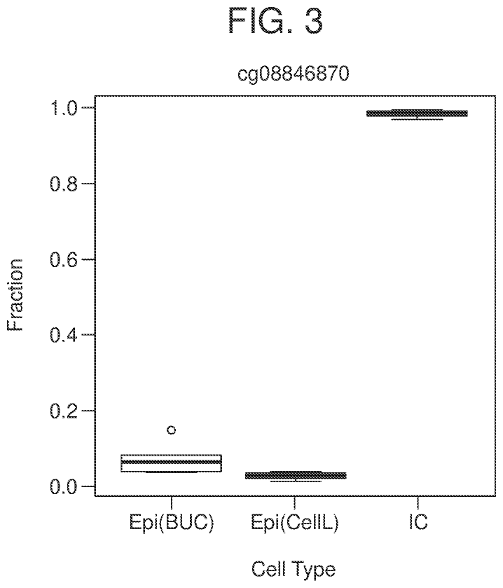

For a CpG to qualify as member of the DMRM we not only require a statistically significant different DNAm between the epithelial and immune-cell samples, but also require at least a 90% methylation difference between the average DNAm levels in both cell-types. This results in an extremely robust DMRM. In summary, the resulting DMRM for saliva consists of 64 CpGs (cg08846870, cg25139229, cg18982286, cg20820767, cg01657186, cg06457736, cg08804626, cg13496041, cg01311222, cg22216196, cg22935422, cg04869379, cg10818657, cg06055229, cg00715197, cg01062942, cg09958560, cg24757533, cg06267617, cg10257110, cg03331514, cg11586930, cg19082559, cg13493526, cg08101036, cg20944964, cg06830450, cg24002183, cg05258935, cg23218363, cg13189207, cg12050271, cg07380416, cg17936488, cg24749672, cg20695297, cg15046675, cg08075204, cg09638208, cg16321975, cg09354037, cg16509569, cg14556909, cg18555277, cg02794695, cg22801799, cg20425130, cg25666403, cg02026204, cg09153080, cg07420137, cg01951274, cg17127769, cg11528914, cg12253437, cg26232412, cg27482619, cg25574765, cg01903374, cg21376733, cg06380123, cg10673833, cg27284288, and cg13165140), 32 of which exhibit close to 100% and 0% methylation in squamous epithelial and immune cells, respectively and with the other 32 exhibiting the exact opposite pattern. See FIGS. 1-3.

Next, we describe the construction of the 8 cell-type DMRM for saliva. This DMRM consists of 2 components, the first one being the 2 cell-type DMRM built earlier. The 2^{nd} component aims to infer the fractions of the 7 different immune cell subtypes, and is built following a similar procedure as described in Zheng S C, Webster A P, Dong D, Feber A, Graham D G, Sullivan R, Jevons S, Lovat L B, Beck S, Widschwendter M, Teschendorff A E: A novel cell-type deconvolution algorithm reveals substantial contamination by immune cells in saliva, buccal and cervix. Epigenomics 2018, 10:925-940. The resulting DMRM consists of 157 CpGs (approximately 23 marker CpGs for each of 7 immune cell types) and 7 immune cell types (CD4+ and CD8+ T-cells, NK-cells, B-cells, Neutrophils, Monocytes and Eosinophils). Those 157CpGs include: cg24462702, cg25599673, cg11944101, cg02661764, cg1040649, cg24662823, cg01874152, cg16193207, cg25486399, cg10240150, cg07713946, cg07960083, cg11848483, cg21224730, cg10864951, cg25226014, cg12032198, cg13750061, cg03274669, cg19994968, cg24612198, cg08450017, cg06164961, cg00219921, cg11531557, cg02324835, cg05356800, cg22999502, cg08622923, cg22512531, cg24456340, cg04759756, cg14094409, cg10977115, cg11583544, cg04042333, cg26518932, cg11804414, cg26757673, cg13988440, cg07728874, cg16039157, cg10480329, cg16636767, cg13781869, cg22675702, cg11664417, cg13430807, cg00208012, cg24788483, cg25517015, cg26326621, cg02780988, cg05923857, cg20641531, cg24365417, cg10278149, cg14976569, cg26997966, cg12655112, cg12873119, cg12207930, cg12037947, cg27366072, cg26538782, cg15035590, cg14936008, cg20452738, cg05205074, cg20602300, cg04838847, cg17232476, cg18664915, cg13823257, cg24777505, cg05579731, cg23683962, cg23730277, cg00867406, cg19276014, cg22281206, cg07721872, cg02087075, cg17304531, cg04289069, cg03730703, cg14060402, cg14331899, cg13617280, cg05875239, cg03538296, cg11335172, cg15956469, cg15241779, cg10628126, cg27309871, cg06202778, cg18310515, cg10142452, cg21474838, cg01940139, cg03605454, cg21333217, cg11100450, cg03708221, cg15052335, cg02150910, cg11637084, cg03886681, cg07307830, cg26227465, cg13468144, cg03146219, cg25600606, cg01040749, cg23599224, cg15520845, cg10611016, cg21792134, cg12809098, cg11597277, cg10487428, cg00084577, cg17080697, cg17932662, cg26105956, cg09163720, cg13079571, cg09859659, cg00471371, cg01718139, cg07356342, cg22381196, cg20720686, cg20240243, cg04476877, cg05078091, cg01953317, cg07050712, cg22543648, cg10454864, cg26103369, cg18966140, cg08077807, cg21510284, cg02494549, cg26156120, cg21261709, cg06620016, cg14320120, cg02031326, cg00059879, cg02195201, cg07033790, cg25270424, cg16760382, and cg20263733.

### 2 Preliminary Validation of the DNA Methylation Reference Matrix for Saliva

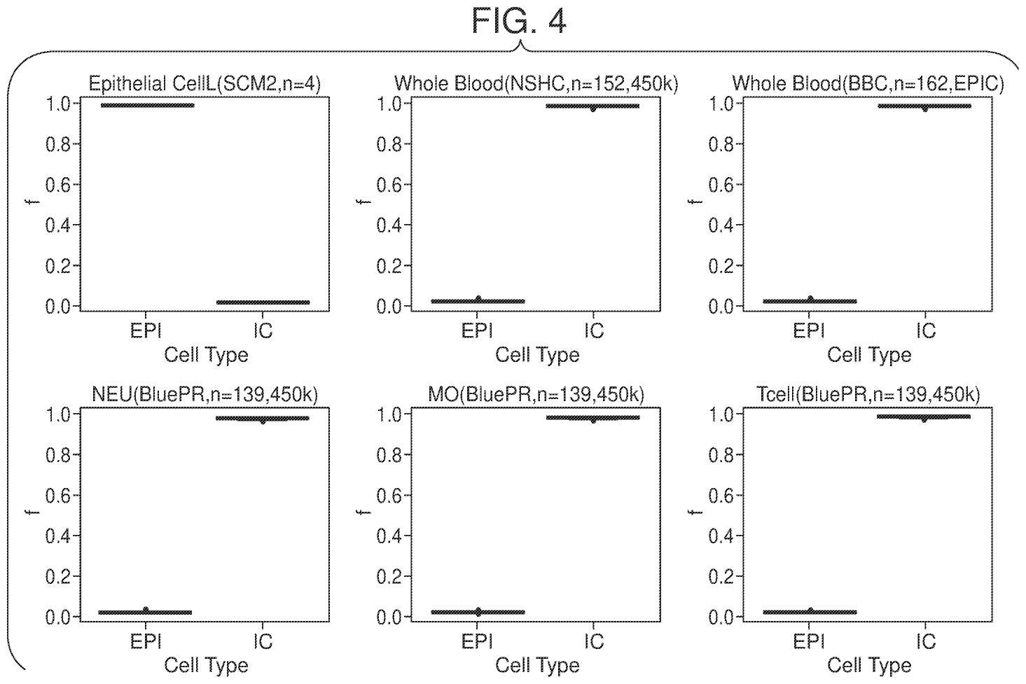

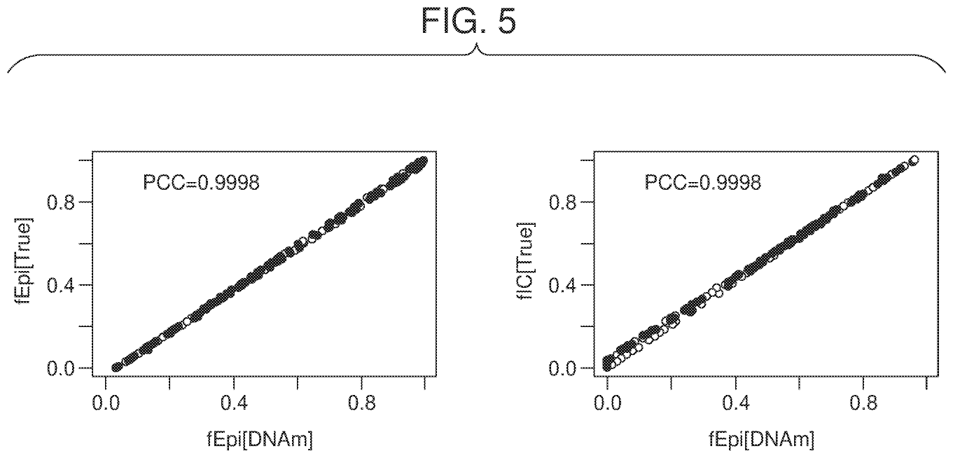

We next provide the data to show that the 2 cell-type DMRM works, by comparing the estimated cell-type fractions derived from it with known cell-type fractions. We provide two levels of validation:
In one analysis, we use the DNAm reference to estimate the cell-type fractions in independent epithelial cell-lines from the Stem-Cell-Matrix Compendium-2 (SCM2) Nazor K L, Altun G, Lynch C, Tran H, Harness J V, Slavin I, Garitaonandia I, Muller F J, Wang Y C, Boscolo F S, et al: Recurrent variations in DNA methylation in human pluripotent stem cells and their differentiated derivatives. Cell Stem Cell 2012, 10:620-634.)((i.e. not used in the DNAm reference construction), in independent whole blood samples from (Teschendorff A E, Yang Z, Wong A, Pipinikas C P, Jiao Y, Jones A, Anjum S, Hardy R, Salvesen H B, Thirlwell C, et al: Correlation of Smoking-Associated DNA Methylation Changes in Buccal Cells With DNA Methylation Changes in Epithelial Cancer. JAMA Oncol 2015, 1:476-485.) (450k technology) and from an internal dataset (EPIC technology), and in independent purified blood cell subtype samples from BLUEPRINT (Chen L, Ge B, Casale F P, Vasquez L, Kwan T, Garrido-Martin D, Watt S, Yan Y, Kundu K, Ecker S, et al: Genetic Drivers of Epigenetic and Transcriptional Variation in Human Immune Cells. Cell 2016, 167:1398-1414 e1324.).
The above procedure is carried out again but on simulated mixtures of the SCM2 epithelial cell-lines and whole blood samples, where the fractions making up the mixtures are known to us by construction. FIGS. 4 and 5 depict the validation results from these analysis.

Together, the above data show that the saliva DMRM defined for 2 cell-types is very accurate in predicting the true cell-type fractions of independent samples. Importantly, what the above data also demonstrates is that the performance is robust to using different technologies. Since the DMRM was built using Illumina 450k profiles, and only about 90% of the CpGs in this matrix will be present in data generated with the EPIC technology, this means that our results are robust to some CpGs in the reference matrix not being present in the validation data.

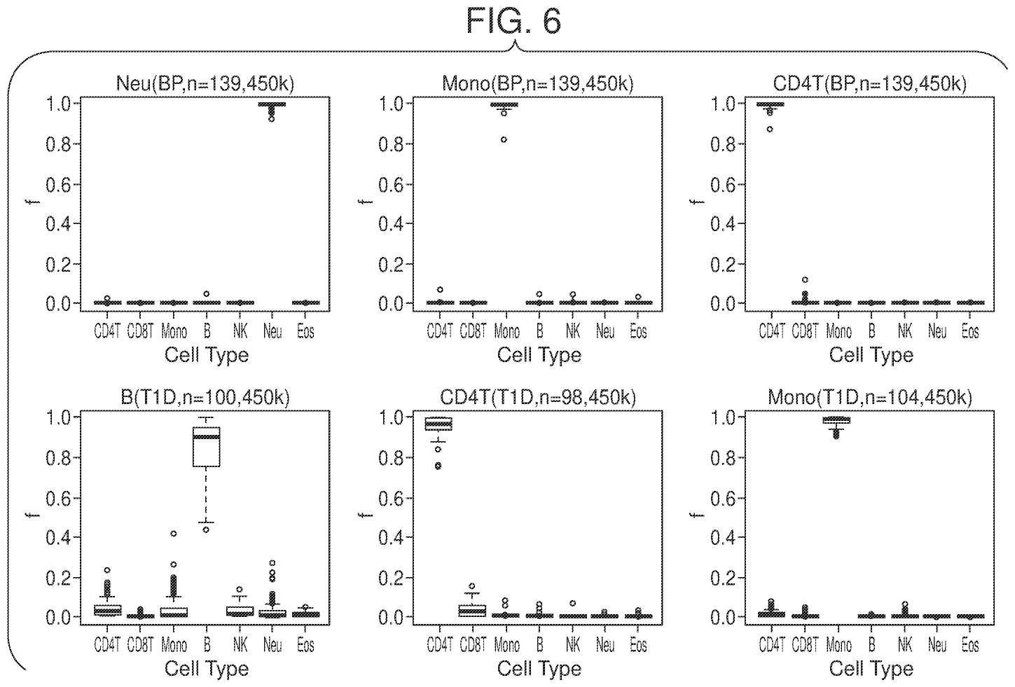

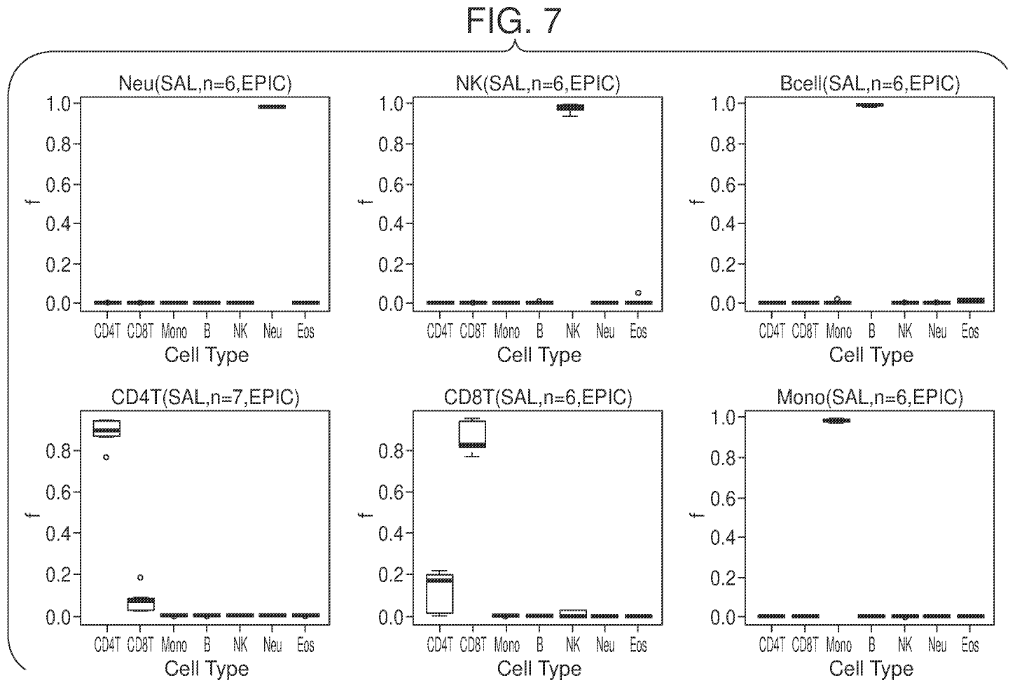

Next, we provide details of the validation of the 8 cell-type DMRM. First we applied it to a number of Illumina 450k/EPIC datasets profiling purified immune cell-type samples (Chen L, Ge B, Casale F P, Vasquez L, Kwan T, Garrido-Martin D, Watt S, Yan Y, Kundu K, Ecker S, et al: Genetic Drivers of Epigenetic and Transcriptional Variation in Human Immune Cells. Cell 2016, 167:1398-1414 e1324./Paul D S, Teschendorff A E, Dang M A, Lowe R, Hawa M I, Ecker S, Beyan H, Cunningham S, Fouts A R, Ramelius A, et al: Increased DNA methylation variability in type 1 diabetes across three immune effector cell types. Nat Commun 2016, 7:13555./Salas L A, Koestler D C, Butler R A, Hansen H M, Wiencke J K, Kelsey K T, Christensen B C: An optimized library for reference-based deconvolution of whole-blood biospecimens assayed using the Illumina HumanMethylationEPIC BeadArray. Genome Biol 2018, 19:64.). FIGS. 6 and 7 illustrate that our 8 cell-type DMRM correctly predicts the high purity of these samples.

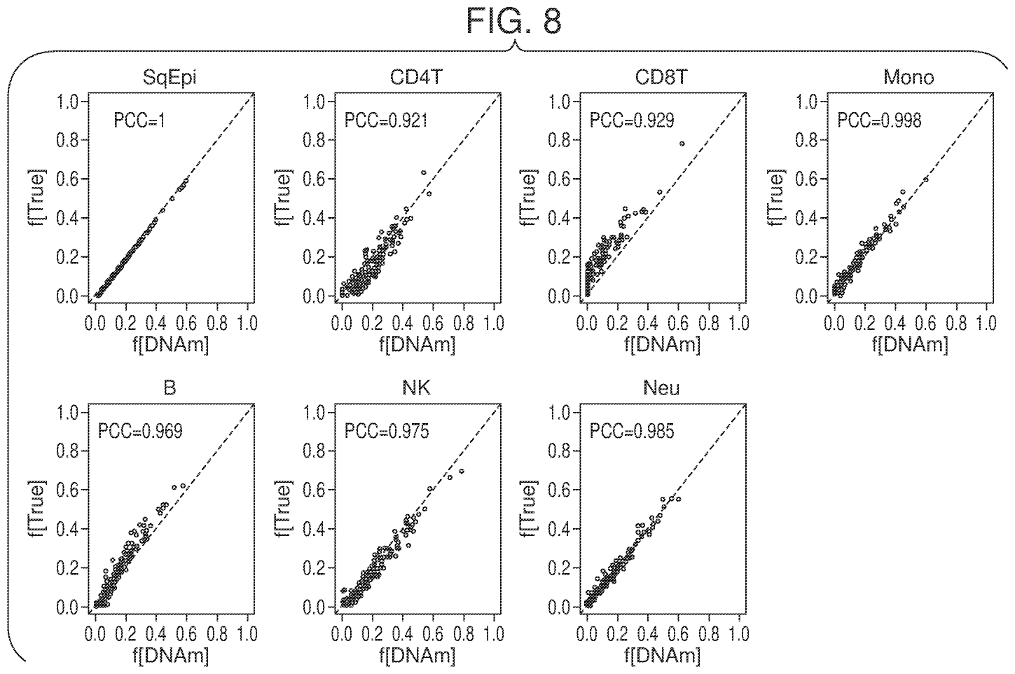

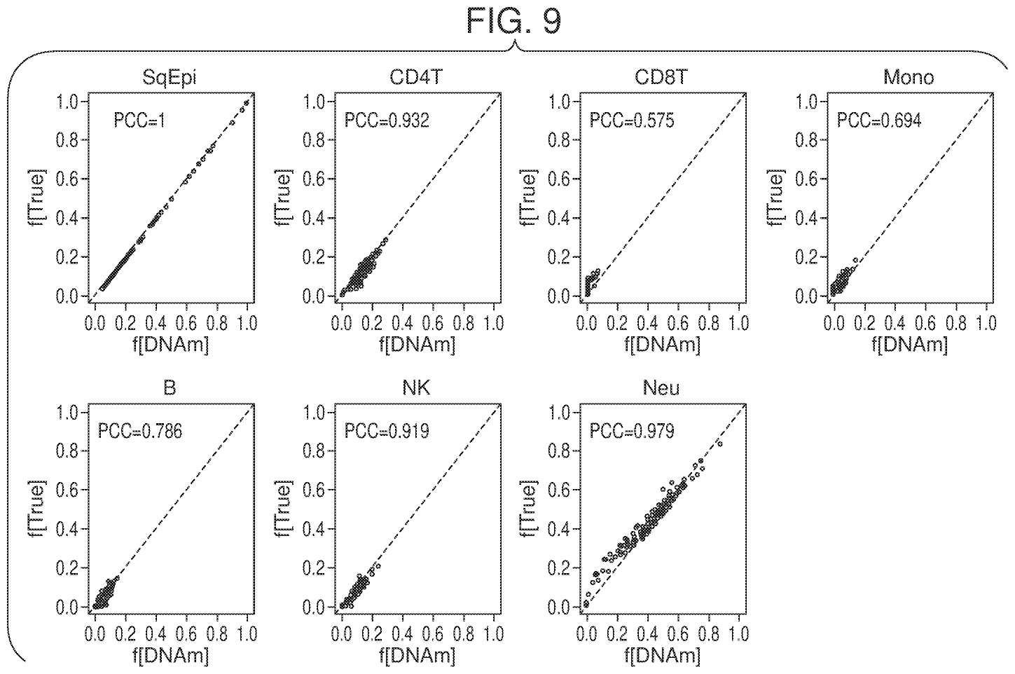

Next, we generated in-silico mixtures (where the mixing proportions are known to us, by construction) to test that our DMRM can predict the correct cell-type fractions. We provide two separate analysis: in the first one, the mixing proportions are chosen uniformly and randomly, so each cell-type is treated as having the same average proportion in saliva. In the second one, mixing proportions are derived from realistic estimates derived from previous saliva and whole blood studies. FIGS. 8 and 9 show that we obtain reasonably good predictions.

The validated two cell-type DNA methylation reference matrix (DMRM) and the validated eight cell type DNA methylation reference matrix DMRM previously described are both useful in methods for determining a percentage of cell-types in a saliva specimen.

For the two cell-type DMRM (epithelial cells and immune cells), the method comprises, consist of or consists essentially of the steps of: (a) obtaining genomic DNA from the saliva specimen, (b) observing cytosine methylation at 64 CG loci, in the genomic DNA of the saliva specimen, wherein said observing includes performing a bisulfate conversion process on the genomic DNA of the saliva specimen so that cytosine residues in the genomic DNA of the human subject are transformed to uracil, while 5-methylcytosine residues in the genomic DNA of the saliva specimen are not transformed to uracil, (c) comparing the CG loci methylation observed in (b) to the CG loci methylation observed in genomic DNA collected from a reference group of saliva specimens and (d) correlating the CG loci methylation observed in (b) with CG loci methylation observed in the reference group of saliva specimens to determine the percentage of cell-types in the saliva specimen wherein the 64 CG loci are cg08846870, cg25139229, cg18982286, cg20820767, cg01657186, cg06457736, cg08804626, cg13496041, cg01311222, cg22216196, cg22935422, cg04869379, cg10818657, cg06055229, cg00715197, cg01062942, cg09958560, cg24757533, cg06267617, cg10257110, cg03331514, cg11586930, cg19082559, cg13493526, cg08101036, cg20944964, cg06830450, cg24002183, cg05258935, cg23218363, cg13189207, cg12050271, cg07380416, cg17936488, cg24749672, cg20695297, cg15046675, cg08075204, cg09638208, cg16321975, cg09354037, cg16509569, cg14556909, cg18555277, cg02794695, cg22801799, cg20425130, cg25666403, cg02026204, cg09153080, cg07420137, cg01951274, cg17127769, cg11528914, cg12253437, cg26232412, cg27482619, cg25574765, cg01903374, cg21376733, cg06380123, cg10673833, cg27284288, and cg13165140.

The obtained genomic DNA from the saliva specimen is derived from a human subject. Similarly, the genomic DNA obtained from the reference group of specimens is derived from a reference group of human individuals. Any number of different saliva collection kits known in the art may be used to collect the saliva of the human subject. Useful kits for this purpose include, but are not necessarily limited to the Oragene DNA OG-500 (DNAGenotek, Ottawa, Canada) kit and the SimplOFy™ (Oasis Diagnostics® Corporation, Vancouver, Canada) kit. In one possible embodiment, the human subject is instructed to fast for a minimum of one hour and then to spit repeatedly into a saliva DNA collection device to a total of 2 mL.

The bisulfate conversion process referenced in step (b) is known in the art. Kits useful for bisulfate conversion are commercially available from a number of manufacturers including Human Genetic Signatures' Methyleasy and Chemicon's CpGenome Modification Kit. See also, WO04096825A1, which describes bisulfite modification methods and Olek et al. Nuc. Acids Res. 24:5064-6 (1994), which discloses methods of performing bisulfite treatment and subsequent amplification.

In at least one possible embodiment of the method, step (b) of observing cytosine methylation at 64 CG loci, in the genomic DNA of the human subject, wherein said observing includes performing a bisulfate conversion process on the genomic DNA of the saliva specimen so that cytosine residues in the genomic DNA of the saliva specimen are transformed to uracil, while 5-methylcytosine residues in the genomic DNA of the saliva specimen are not transformed to uracil includes a number of additional steps. More specifically, the performing of the bisulfate conversion process may include the steps of amplifying the genomic DNA of the saliva specimen by a polymerase chain reaction process, hybridizing the genomic DNA to a beadchip to create a genomic DNA hybridized beadchip, staining the genomic DNA hybridized beadchip and then imaging the genomic DNA hybridized beadchip.

The imaging of the genomic DNA hybridized beadchip may be performed using the IIlumina EPIC850k array which provides for unambiguous CpG loci identification for purposes of observing methylation at specific CpG loci of the human DNA genome. Alternatively, other methods of epigenetic beta value collection may be used if desired.

Still further, the method may include using a regression algorithm to correlate the CG loci methylation observed in (b) with the CG loci methylation observed in the reference group of saliva specimens of known cell-type percentages. This allows for good correlation between the CG loci methylation observed in (b) with the CG loci methylation observed in the reference group of saliva specimens resulting in a better or more accurate determination of the percentage of cell-types in a saliva specimen.

For the eight cell-type DMRM (epithelial cells, and the immune cell types: neutrophils, eosinophils, monocytes, CD4+ and CD8+ T-cells, natural (NK) killer cells and B-cells), the method comprises, consist of or consists essentially of the steps of: (a) obtaining genomic DNA from the saliva specimen, (b) observing cytosine methylation at 157 CG loci, in the genomic DNA of the saliva specimen, wherein said observing includes performing a bisulfate conversion process on the genomic DNA of the saliva specimen so that cytosine residues in the genomic DNA of the saliva specimen are transformed to uracil, while 5-methylcytosine residues in the genomic DNA of the saliva specimen are not transformed to uracil, (c) comparing the CG loci methylation observed in (b) to the CG loci methylation observed in genomic DNA collected from a reference group of saliva specimens and (d) correlating the CG loci methylation observed in (b) with CG loci methylation observed in the reference group of saliva specimens to determine the biological age of the human subject wherein the 157 CG loci are cg24462702, cg25599673, cg11944101, cg02661764, cg1040649, cg24662823, cg01874152, cg16193207, cg25486399, cg10240150, cg07713946, cg07960083, cg11848483, cg21224730, cg10864951, cg25226014, cg12032198, cg13750061, cg03274669, cg19994968, cg24612198, cg08450017, cg06164961, cg00219921, cg11531557, cg02324835, cg05356800, cg22999502, cg08622923, cg22512531, cg24456340, cg04759756, cg14094409, cg10977115, cg11583544, cg04042333, cg26518932, cg11804414, cg26757673, cg13988440, cg07728874, cg16039157, cg10480329, cg16636767, cg13781869, cg22675702, cg11664417, cg13430807, cg00208012, cg24788483, cg25517015, cg26326621, cg02780988, cg05923857, cg20641531, cg24365417, cg10278149, cg14976569, cg26997966, cg12655112, cg12873119, cg12207930, cg12037947, cg27366072, cg26538782, cg15035590, cg14936008, cg20452738, cg05205074, cg20602300, cg04838847, cg17232476, cg18664915, cg13823257, cg24777505, cg05579731, cg23683962, cg23730277, cg00867406, cg19276014, cg22281206, cg07721872, cg02087075, cg17304531, cg04289069, cg03730703, cg14060402, cg14331899, cg13617280, cg05875239, cg03538296, cg11335172, cg15956469, cg15241779, cg10628126, cg27309871, cg06202778, cg18310515, cg10142452, cg21474838, cg01940139, cg03605454, cg21333217, cg11100450, cg03708221, cg15052335, cg02150910, cg11637084, cg03886681, cg07307830, cg26227465, cg13468144, cg03146219, cg25600606, cg01040749, cg23599224, cg15520845, cg10611016, cg21792134, cg12809098, cg11597277, cg10487428, cg00084577, cg17080697, cg17932662, cg26105956, cg09163720, cg13079571, cg09859659, cg00471371, cg01718139, cg07356342, cg22381196, cg20720686, cg20240243, cg04476877, cg05078091, cg01953317, cg07050712, cg22543648, cg10454864, cg26103369, cg18966140, cg08077807, cg21510284, cg02494549, cg26156120, cg21261709, cg06620016, cg14320120, cg02031326, cg00059879, cg02195201, cg07033790, cg25270424, cg16760382, and cg20263733.

As with the two cell-type DMRM, the obtained genomic DNA from the saliva specimen is derived from a human subject. Similarly, the genomic DNA obtained from the reference group of saliva specimens is derived from a reference group of human individuals. Any number of different saliva collection kits known in the art may be used to collect the saliva of the human subject. Useful kits for this purpose include, but are not necessarily limited to the Oragene DNA OG-500 (DNAGenotek, Ottawa, Canada) kit and the SimplOFy™ (Oasis Diagnostics® Corporation, Vancouver, Canada) kit. In one possible embodiment, the human subject is instructed to fast for a minimum of one hour and then to spit repeatedly into a saliva DNA collection device to a total of 2 mL.

The bisulfate conversion process referenced in step (b) is known in the art. Kits useful for bisulfate conversion are commercially available from a number of manufacturers including Human Genetic Signatures' Methyleasy and Chemicon's CpGenome Modification Kit. See also, WO04096825A1, which describes bisulfite modification methods and Olek et al. Nuc. Acids Res. 24:5064-6 (1994), which discloses methods of performing bisulfite treatment and subsequent amplification.

In at least one possible embodiment of the method, step (b) of observing cytosine methylation at 157 CG loci, in the genomic DNA of the saliva specimen, wherein said observing includes performing a bisulfate conversion process on the genomic DNA of the saliva specimen so that cytosine residues in the genomic DNA of the saliva specimen are transformed to uracil, while 5-methylcytosine residues in the genomic DNA of the saliva specimen are not transformed to uracil includes a number of additional steps. More specifically, the performing of the bisulfate conversion process may include the steps of amplifying the genomic DNA of the saliva specimen by a polymerase chain reaction process, hybridizing the genomic DNA to a beadchip to create a genomic DNA hybridized beadchip, staining the genomic DNA hybridized beadchip and then imaging the genomic DNA hybridized beadchip.

The imaging of the genomic DNA hybridized beadchip may be performed using the Illumina EPIC850k array which provides for unambiguous CpG loci identification for purposes of observing methylation at specific CpG loci of the human DNA genome. Alternatively, other methods of epigenetic beta value collection may be used if desired.

Still further, the method may include using a regression algorithm to correlate the CG loci methylation observed in (b) with the CG loci methylation observed in the reference group of saliva specimens of known cell-type percentages. This allows for good correlation between the CG loci methylation observed in (b) with the CG loci methylation observed in the reference group of saliva specimens resulting in a better or more accurate determination of the biological age of the human subject.

This disclosure may be considered to relate to the following items.

1. A method of determining a percentage of cell-types in a saliva specimen, comprising:
(a) obtaining genomic DNA from the saliva specimen;
(b) observing cytosine methylation at 157 CG loci, in the genomic DNA of the saliva specimen, wherein said observing includes performing a bisulfate conversion process on the genomic DNA of the saliva specimen so that cytosine residues in the genomic DNA of the saliva specimen are transformed to uracil, while 5-methylcytosine residues in the genomic DNA of the saliva specimen are not transformed to uracil;
(c) comparing the CG loci methylation observed in (b) to the CG loci methylation observed in genomic DNA of a reference group of saliva specimens; and
(d) correlating the CG loci methylation observed in (b) with CG loci methylation observed in the reference group of saliva specimens to determine the percentage of cell-types in the saliva specimen wherein the 157 CG loci are cg24462702, cg25599673, cg11944101, cg02661764, cg1040649, cg24662823, cg01874152, cg16193207, cg25486399, cg10240150, cg07713946, cg07960083, cg11848483, cg21224730, cg10864951, cg25226014, cg12032198, cg13750061, cg03274669, cg19994968, cg24612198, cg08450017, cg06164961, cg00219921, cg11531557, cg02324835, cg05356800, cg22999502, cg08622923, cg22512531, cg24456340, cg04759756, cg14094409, cg10977115, cg11583544, cg04042333, cg26518932, cg11804414, cg26757673, cg13988440, cg07728874, cg16039157, cg10480329, cg16636767, cg13781869, cg22675702, cg11664417, cg13430807, cg00208012, cg24788483, cg25517015, cg26326621, cg02780988, cg05923857, cg20641531, cg24365417, cg10278149, cg14976569, cg26997966, cg12655112, cg12873119, cg12207930, cg12037947, cg27366072, cg26538782, cg15035590, cg14936008, cg20452738, cg05205074, cg20602300, cg04838847, cg17232476, cg18664915, cg13823257, cg24777505, cg05579731, cg23683962, cg23730277, cg00867406, cg19276014, cg22281206, cg07721872, cg02087075, cg17304531, cg04289069, cg03730703, cg14060402, cg14331899, cg13617280, cg05875239, cg03538296, cg11335172, cg15956469, cg15241779, cg10628126, cg27309871, cg06202778, cg18310515, cg10142452, cg21474838, cg01940139, cg03605454, cg21333217, cg11100450, cg03708221, cg15052335, cg02150910, cg11637084, cg03886681, cg07307830, cg26227465, cg13468144, cg03146219, cg25600606, cg01040749, cg23599224, cg15520845, cg10611016, cg21792134, cg12809098, cg11597277, cg10487428, cg00084577, cg17080697, cg17932662, cg26105956, cg09163720, cg13079571, cg09859659, cg00471371, cg01718139, cg07356342, cg22381196, cg20720686, cg20240243, cg04476877, cg05078091, cg01953317, cg07050712, cg22543648, cg10454864, cg26103369, cg18966140, cg08077807, cg21510284, cg02494549, cg26156120, cg21261709, cg06620016, cg14320120, cg02031326, cg00059879, cg02195201, cg07033790, cg25270424, cg16760382, and cg20263733.

2. The method of item 1, including amplifying the genomic DNA of the saliva specimen by a polymerase chain reaction process.

3. The method of item 2, wherein the observing of the cytosine methylation at the 157 CG loci includes hybridizing the genomic DNA to a beadchip to create a genomic DNA hybridized beadchip, staining the genomic DNA hybridized beadchip and then imaging the genomic DNA hybridized beadchip.

4. The method of item 3, including using a regression algorithm to correlate the CG loci methylation observed in (b) with the CG loci methylation observed in the reference group of saliva specimens.

5. The method of item 4, wherein the saliva specimen is derived from a human subject.

6. The method of item 5, wherein the genomic DNA of the reference group of saliva specimens is derived from a reference group of human individuals.

7. The method of item 1, wherein the observing of the cytosine methylation at the 157 CG loci includes hybridizing the genomic DNA to a beadchip to create a genomic DNA hybridized beadchip, staining the genomic DNA hybridized beadchip and then imaging the genomic DNA hybridized beadchip.

8. The method of item 1, including using a regression algorithm to correlate the CG loci methylation observed in (b) with the CG loci methylation observed in the reference group of saliva specimens.

9. The method of item 1, wherein the genomic DNA of the saliva specimen is derived from a human subject.

10. The method of item 9, wherein the genomic DNA of the reference group of saliva specimens is derived from a reference group of human individuals.

11. A method of determining a percentage of cell-types in a saliva specimen, comprising:
(a) obtaining genomic DNA from the saliva specimen;
(b) observing cytosine methylation at 64 CG loci, in the genomic DNA of the saliva specimen, wherein said observing includes performing a bisulfate conversion process on the genomic DNA of the saliva specimen so that cytosine residues in the genomic DNA of the saliva specimen are transformed to uracil, while 5-methylcytosine residues in the genomic DNA of the saliva are not transformed to uracil;
(c) comparing the CG loci methylation observed in (b) to the CG loci methylation observed in genomic DNA collected from a reference group of saliva specimens; and
(d) correlating the CG loci methylation observed in (b) with CG loci methylation observed in the reference group of saliva specimens to determine the percentage of cell-types in the saliva specimen wherein the 64 CG loci are cg08846870, cg25139229, cg18982286, cg20820767, cg01657186, cg06457736, cg08804626, cg13496041, cg01311222, cg22216196, cg22935422, cg04869379, cg10818657, cg06055229, cg00715197, cg01062942, cg09958560, cg24757533, cg06267617, cg10257110, cg03331514, cg11586930, cg19082559, cg13493526, cg08101036, cg20944964, cg06830450, cg24002183, cg05258935, cg23218363, cg13189207, cg12050271, cg07380416, cg17936488, cg24749672, cg20695297, cg15046675, cg08075204, cg09638208, cg16321975, cg09354037, cg16509569, cg14556909, cg18555277, cg02794695, cg22801799, cg20425130, cg25666403, cg02026204, cg09153080, cg07420137, cg01951274, cg17127769, cg11528914, cg12253437, cg26232412, cg27482619, cg25574765, cg01903374, cg21376733, cg06380123, cg10673833, cg27284288, and cg13165140.

12. The method of item 11, including amplifying the genomic DNA of the saliva specimen by a polymerase chain reaction process.

13. The method of item 12, wherein the observing of the cytosine methylation at the 64 CG loci includes hybridizing the genomic DNA to a beadchip to create a genomic DNA hybridized beadchip, staining the genomic DNA hybridized beadchip and then imaging the genomic DNA hybridized beadchip.

14. The method of item 13, including using a regression algorithm to correlate the CG loci methylation observed in (b) with the CG loci methylation observed in the reference group of saliva specimens.

15. The method of item 14, wherein the genomic DNA of the saliva specimen is derived from a human subject.

16. The method of item 15, wherein the genomic DNA of the reference group of saliva specimens is derived from a reference group of human individuals.

17. The method of item 11, wherein the observing of the cytosine methylation at the 64 CG loci includes hybridizing the genomic DNA to a beadchip to create a genomic DNA hybridized beadchip, staining the genomic DNA hybridized beadchip and then imaging the genomic DNA hybridized beadchip.

18. The method of item 11, including using a regression algorithm to correlate the CG loci methylation observed in (b) with the CG loci methylation observed in the reference group of saliva specimens.

19. The method of claim 11, wherein the genomic DNA of the saliva specimen is derived from a human subject.

20. The method of item 19, wherein the genomic DNA of the reference group of saliva specimens is derived from a reference group of human individuals.

Each of the following terms written in singular grammatical form: “a”, “an”, and the”, as used herein, means “at least one”, or “one or more”. Use of the phrase “One or more” herein does not alter this intended meaning of “a”, “an”, or “the”. Accordingly, the terms “a”, “an”, and “the”, as used herein, may also refer to, and encompass, a plurality of the stated entity or object, unless otherwise specifically defined or stated herein, or, unless the context clearly dictates otherwise. For example, the phrases: “a beadchip” and “a step or procedure”, as used herein, may also refer to, and encompass, a plurality of beadchips and a plurality of steps or procedures, respectively.

Each of the following terms: “includes”, “including”, “has”, “having”, “comprises”, and “comprising”, and, their linguistic/grammatical variants, derivatives, or/and conjugates, as used herein, means “including, but not limited to”, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof.

The phrase “consisting of”, as used herein, is closed-ended and excludes any element, step, or ingredient not specifically mentioned. The phrase “consisting essentially of”, as used herein, is a semi-closed term indicating that an item is limited to the components specified and those that do not materially affect

the basic and novel characteristic(s) of what is specified.

It is to be fully understood that certain aspects, characteristics, and features, of the method of determining the biological age of a human subject, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the method which are illustratively described and presented in combination or sub-combination in the context or format of a single embodiment may also be illustratively described and presented in the context or format of a plurality of separate embodiments.

Although the method of determining the biological age of a human subject have been illustratively described and presented by way of specific exemplary embodiments, and examples thereof, it is evident that many alternatives, modifications, or/and variations, thereof, will be apparent to those skilled in the art. Accordingly, it is intended that all such alternatives, modifications, or/and variations, fall within the spirit of, and are encompassed by, the broad scope of the appended claims.

What is claimed is:

1. A method of determining a percentage of cell-types in a saliva specimen, comprising:
(a) obtaining genomic DNA from the saliva specimen;
(b) observing cytosine methylation at 157 CG loci, in the genomic DNA of the saliva specimen, wherein said observing includes performing a bisulfate conversion process on the genomic DNA of the saliva specimen so that cytosine residues in the genomic DNA of the saliva specimen are transformed to uracil, while 5-methylcytosine residues in the genomic DNA of the saliva specimen are not transformed to uracil;
(c) comparing the CG loci methylation observed in (b) to the CG loci methylation observed in genomic DNA of a reference group of saliva specimens; and
(d) correlating the CG loci methylation observed in (b) with CG loci methylation observed in the reference group of saliva specimens to determine the percentage of cell-types in the saliva specimen wherein the 157 CG loci are cg24462702, cg25599673, cg11944101, cg02661764, cg1040649, cg24662823, cg01874152, cg16193207, cg25486399, cg10240150, cg07713946, cg07960083, cg11848483, cg21224730, cg10864951, cg25226014, cg12032198, cg13750061, cg03274669, cg19994968, cg24612198, cg08450017, cg06164961, cg00219921, cg11531557, cg02324835, cg05356800, cg22999502, cg08622923, cg22512531, cg24456340, cg04759756, cg14094409, cg10977115, cg11583544, cg04042333, cg26518932, cg11804414, cg26757673, cg13988440, cg07728874, cg16039157, cg10480329, cg16636767, cg13781869, cg22675702, cg11664417, cg13430807, cg00208012, cg24788483, cg25517015, cg26326621, cg02780988, cg05923857, cg20641531, cg24365417, cg10278149, cg14976569, cg26997966, cg12655112, cg12873119, cg12207930, cg12037947, cg27366072, cg26538782, cg15035590, cg14936008, cg20452738, cg05205074, cg20602300, cg04838847, cg17232476, cg18664915, cg13823257, cg24777505, cg05579731, cg23683962, cg23730277, cg00867406, cg19276014, cg22281206, cg07721872, cg02087075, cg17304531, cg04289069, cg03730703, cg14060402, cg14331899, cg13617280, cg05875239, cg03538296, cg11335172, cg15956469, cg15241779, cg10628126, cg27309871, cg06202778, cg18310515, cg10142452, cg21474838, cg01940139, cg03605454, cg21333217, cg11100450, cg03708221, cg15052335, cg02150910, cg11637084, cg03886681, cg07307830, cg26227465, cg13468144, cg03146219, cg25600606, cg01040749, cg23599224, cg15520845, cg10611016, cg21792134, cg12809098, cg11597277, cg10487428, cg00084577, cg17080697, cg17932662, cg26105956, cg09163720, cg13079571, cg09859659, cg00471371, cg01718139, cg07356342, cg22381196, cg20720686, cg20240243, cg04476877, cg05078091, cg01953317, cg07050712, cg22543648, cg10454864, cg26103369, cg18966140, cg08077807, cg21510284, cg02494549, cg26156120, cg21261709, cg06620016, cg14320120, cg02031326, cg00059879, cg02195201, cg07033790, cg25270424, cg16760382, and cg20263733.

2. The method of claim 1, including amplifying the genomic DNA of the saliva specimen by a polymerase chain reaction process.

3. The method of claim 2, wherein the observing of the cytosine methylation at the 157 CG loci includes hybridizing the genomic DNA to a beadchip to create a genomic DNA hybridized beadchip, staining the genomic DNA hybridized beadchip and then imaging the genomic DNA hybridized beadchip.

4. The method of claim 3, including using a regression algorithm to correlate the CG loci methylation observed in (b) with the CG loci methylation observed in the reference group of saliva specimens.

5. The method of claim 4, wherein the saliva specimen is derived from a human subject.

6. The method of claim 5, wherein the genomic DNA of the reference group of saliva specimens is derived from a reference group of human individuals.

7. The method of claim 1, wherein the observing of the cytosine methylation at the 157 CG loci includes hybridizing the genomic DNA to a beadchip to create a genomic DNA hybridized beadchip, staining the genomic DNA hybridized beadchip and then imaging the genomic DNA hybridized beadchip.

8. The method of claim 1, including using a regression algorithm to correlate the CG loci methylation observed in (b) with the CG loci methylation observed in the reference group of saliva specimens.

9. The method of claim 1, wherein the genomic DNA of the saliva specimen is derived from a human subject.

10. The method of claim 9, wherein the genomic DNA of the reference group of saliva specimens is derived from a reference group of human individuals.

11. A method of determining a percentage of cell-types in a saliva specimen, comprising:
(a) obtaining genomic DNA from the saliva specimen;
(b) observing cytosine methylation at 64 CG loci, in the genomic DNA of the saliva specimen, wherein said observing includes performing a bisulfate conversion process on the genomic DNA of the saliva specimen so that cytosine residues in the genomic DNA of the saliva specimen are transformed to uracil, while 5-methylcytosine residues in the genomic DNA of the saliva are not transformed to uracil;
(c) comparing the CG loci methylation observed in (b) to the CG loci methylation observed in genomic DNA collected from a reference group of saliva specimens; and
(d) correlating the CG loci methylation observed in (b) with CG loci methylation observed in the reference group of saliva specimens to determine the percentage of cell-types in the saliva specimen wherein the 64 CG loci are cg08846870, cg25139229, cg18982286, cg20820767, cg01657186, cg06457736, cg08804626, cg13496041, cg01311222, cg22216196, cg22935422, cg04869379, cg10818657, cg06055229, cg00715197, cg01062942, cg09958560, cg24757533, cg06267617, cg10257110, cg03331514, cg11586930, cg19082559, cg13493526, cg08101036, cg20944964, cg06830450, cg24002183, cg05258935, cg23218363, cg13189207, cg12050271, cg07380416, cg17936488, cg24749672, cg20695297, cg15046675, cg08075204, cg09638208, cg16321975, cg09354037, cg16509569, cg14556909, cg18555277, cg02794695, cg22801799, cg20425130, cg25666403, cg02026204, cg09153080, cg07420137, cg01951274, cg17127769, cg11528914, cg12253437, cg26232412, cg27482619, cg25574765, cg01903374, cg21376733, cg06380123, cg10673833, cg27284288, and cg13165140.

12. The method of claim 11, including amplifying the genomic DNA of the saliva specimen by a polymerase chain reaction process.

13. The method of claim 12, wherein the observing of the cytosine methylation at the 64 CG loci includes hybridizing the genomic DNA to a beadchip to create a genomic DNA hybridized beadchip, staining the genomic DNA hybridized beadchip and then imaging the genomic DNA hybridized beadchip.

14. The method of claim 13, including using a regression algorithm to correlate the CG loci methylation observed in (b) with the CG loci methylation observed in the reference group of saliva specimens.

15. The method of claim 14, wherein the genomic DNA of the saliva specimen is derived from a human subject.

16. The method of claim 15, wherein the genomic DNA of the reference group of saliva specimens is derived from a reference group of human individuals.

17. The method of claim 11, wherein the observing of the cytosine methylation at the 64 CG loci includes hybridizing the genomic DNA to a beadchip to create a genomic DNA hybridized beadchip, staining the genomic DNA hybridized beadchip and then imaging the genomic DNA hybridized beadchip.

18. The method of claim 11, including using a regression algorithm to correlate the CG loci methylation observed in (b) with the CG loci methylation observed in the reference group of saliva specimens.

19. The method of claim 11, wherein the genomic DNA of the saliva specimen is derived from a human subject.

20. The method of claim 19, wherein the genomic DNA of the reference group of saliva specimens is derived from a reference group of human individuals.
